(19) United States
(12) Patent Application Publication
(10) Pub. No.: US 2024/0189406 A1
(43) Pub. Date: Jun. 13, 2024

(54) NATURAL KILLER T CELL LIGAND LOADED EXTRACELLULAR NANOVESICLE AND AUTOLOGOUS ANTI-CANCER VACCINE FOR HEMATOLOGIC MALIGNANCIES COMPRISING THE SAME

(71) Applicant: SEOUL NATIONAL UNIVERSITY R&DB FOUNDATION, Seoul (KR)

(72) Inventors: Yeonseok CHUNG, Seoul (KR); Suyoung LEE, Seongnam-si (KR); Da-Sol KUEN, Seoul (KR); Byung-Soo KIM, Seoul (KR); Jihye HONG, Anyang-si (KR); Byung-Sik CHO, Seoul (KR)

(73) Assignee: SEOUL NATIONAL UNIVERSITY R&DB FOUNDATION, Seoul (KR)

(21) Appl. No.: 18/528,327

(22) Filed: Dec. 4, 2023

(30) Foreign Application Priority Data
Dec. 8, 2022 (KR) 10-2022-0170963
Nov. 2, 2023 (KR) 10-2023-0150336

(51) Int. Cl. A61K 39/00; A61K 45/06; A61P 35/02

(52) U.S. Cl. CPC A61K 39/001129; A61K 45/06; A61P 35/02; A61K 2039/55555; A61K 2039/804

(57) ABSTRACT

The present invention provides a natural killer T cell ligand-loaded extracellular nanovesicle, and an autologous anticancer vaccine for hematologic malignancies including the above extracellular nanovesicle. More particularly, a cancer cell-derived extracellular nanovesicle in which a natural killer T cell ligand as an adjuvant is bound to a surface thereof, and an autologous anticancer vaccine including the same, which can recognize cancer antigens specific to patients with hematologic malignancies and has cancer cell-specific anticancer function thus to enable T cell activation.

### CROSS-REFERENCE TO RELATED APPLICATIONS

This application claims priority to Korean Patent Application No. 10-2022-0170963 filed Dec. 8, 2022 and Korean Patent Application No. 10-2023-0150336 filed Nov. 2, 2023, the entire disclosures of which are incorporated herein by reference.

### BACKGROUND OF THE INVENTION

### 1. Field of the Invention

The present invention relates to a natural killer T cell ligand-loaded extracellular nanovesicle, and use thereof.

### 2. Description of the Related Art

Hematologic malignancies (“blood cancer”) are cancer occurring in the blood and lymphatic system. They may occur in hematopoietic tissues (for example, bone marrow) or immune cells. The types of blood cancer include acute myeloid leukemia (AML), multiple myeloma (MM), T-cell lymphoma (TCL) and the like.

For example, acute myeloid leukemia is a malignant disease in the bone marrow, characterized by the accumulation of myeloid precursor cells arrested in their early stage of differentiation. Currently, the standard treatment consists of chemotherapy, followed by a high-dose therapy or maintenance reinforcing chemical therapy, which is performed in combination with autologous or allogeneic haemopoietic stem cell transplantation (alloSCT). While the five (5)-year survival rate for patients under the age of 65 who received treatment can range from 40 to 45%, for patients older than 65, the five (5)-year survival rate comes up to only 10%. Therefore, the above disease is known to be difficult to treat.

Although the blood cancer cells express MHC class I wherein T cells can recognize a cancer antigen, it does not provide a signal required for cell activation. Therefore, in many cases, the blood cancer cannot activate T cell response. Further, the blood cancer involves diverse variations and entails a problem of difficult identification of cancer antigen for each patient. Therefore, it is now required to develop a personalized vaccine for blood cancer wherein intrinsic cancer antigens of the blood cancer can be recognized and activated by T cells.

### PRIOR ART DOCUMENT

### Patent Document

Korean Patent Laid-Open Publication No. 2017-013283

### SUMMARY OF THE INVENTION

An object of the present invention is to provide an extracellular nanovesicle (ECNV), which is loaded with natural killer T cell ligand (NKT Ligand) and is derived from cancer cells.

Another object of the present invention is to provide an autologous anticancer vaccine for hematologic malignancies (hereinafter, referred to as “blood cancer”), which includes natural killer T cell ligand-loaded extracellular nanovesicle (NKT Ligand-ECNV) derived from cancer cells.

Further, another object of the present invention is to provide a method for treatment of blood cancer, which includes administering natural killer T cell ligand-loaded extracellular nanovesicle (NKT Ligand-ECNV) derived from cancer cells to an individual.

To achieve the above objects, the following technical solutions are adopted in the present invention.
1. An anticancer vaccine method for hematologic malignancies (“blood cancer”), including a cancer cell-derived extracellular nanovesicle (ECNV), wherein natural killer T cell ligand is bound to the surface of the ECNV.
2. The anticancer vaccine according to the above 1, wherein the natural killer T cell ligand is any one selected from the group consisting of alpha-galactosyl ceramide, alpha-glucuronosyl ceramide, phosphatidylinositol tetramannoside, isoglobo trihexosyl ceramide, ganglioside GD3, phosphatidylcholin, beta-galactosyl ceramide, lipophosphoglycan, glycoinositol phospholipid, beta-anomer galactosyl ceramide and alpha-anomer galactosyl ceramide.
3. The anticancer vaccine according to the above 1, wherein the cancer cell is derived from an individual suffering from any one disease selected from the group consisting of acute myeloid leukemia (AML), acute lymphoblastic leukemia (ALL), chronic myeloid leukemia (CML), chronic lymphoblastic leukemia (CLL), multiple myeloma (MM), B-cell lymphoma (BCL) and T-cell lymphoma (TCL).
4. The anticancer vaccine according to the above 1, wherein the blood cancer is any one selected from the group consisting of acute myeloid leukemia (AML), acute lymphoblastic leukemia (ALL), chronic myeloid leukemia (CML), chronic lymphoblastic leukemia (CLL), multiple myeloma (MM), B-cell lymphoma (BCL) and T-cell lymphoma (TCL).
5. The anticancer vaccine according to the above 1, wherein the extracellular nanovesicle includes a cancer antigen.
6. The anticancer vaccine according to the above 1, wherein the natural killer T cell ligand is bound to CD1d receptor on the surface.
7. The anticancer vaccine according to the above 1, wherein the cancer cell is an autologous cancer cell derived from the individual.
8. The anticancer vaccine according to the above 1, wherein the extracellular nanovesicle is an exosome or micro-vesicle having a size of 100 to 1,000 nm.
9. The anticancer vaccine according to the above 1, wherein the anticancer vaccine is administered in combination with any one selected from the group consisting of a chemotherapy, a target therapeutic agent and an immune checkpoint inhibitor.

The autologous anticancer vaccine of the present invention exhibits excellent therapeutic effects against hematologic malignances (“blood cancer”). Unlike other solid cancers, cancer cells of the blood cancer are highly like to directly interact with T cells, whereby the autologous anticancer vaccine of the present invention may express better effects.

The autologous anticancer vaccine of the present invention is a patient-customized (“personalized”) immune therapeutic agent.

The autologous anticancer vaccine of the present invention may activate natural killer T cells and cancer-specific T cells, simultaneously.

The autologous anticancer vaccine of the present invention may induce innate and adaptive immune responses at the same time.

The autologous anticancer vaccine of the present invention has low toxicity and is safe.

The autologous anticancer vaccine of the present invention does not use cancer cells themselves and is thus safe.

The autologous anticancer vaccine of the present invention can be administered in combination with a chemotherapy, a targeted therapeutic agent and an immunotherapeutic anticancer agent. The chemotherapy and targeted therapy need repeated treatment several times. Further, in most cases, since leucopenia occurs during treatment, the treatment should be paused and then resumed repeatedly. However, the autologous anticancer vaccine of the present invention is a safe strategy that induces blood cancer-specific autologous immune response at a time when the absolute number of leukocytes is recovered, such that it can be used in combination with existing care treatment methods.

Since the autologous anticancer vaccine of the present invention includes cancer cell-derived extracellular nanovesicles, it needs neither identification of cancer antigens nor discrimination of patient-specific neoantigen.

The autologous anticancer vaccine of the present invention may be rapidly manufactured within 1 to 2 days.

The cancer cell-derived extracellular nanovesicle of the present invention is easily produced, compared to exosomes. Further, since a process of identifying a cancer antigen is omitted, a production time may be considerably shortened.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic view illustrating the principle of action of one example of an anticancer vaccine of the present invention;

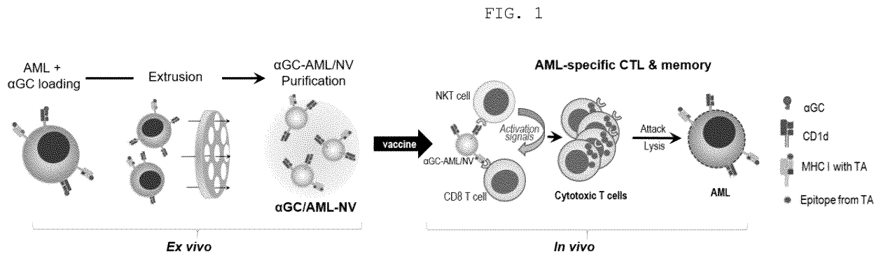

FIG. 2 illustrates the mechanism of action of the anticancer vaccine of the present invention;

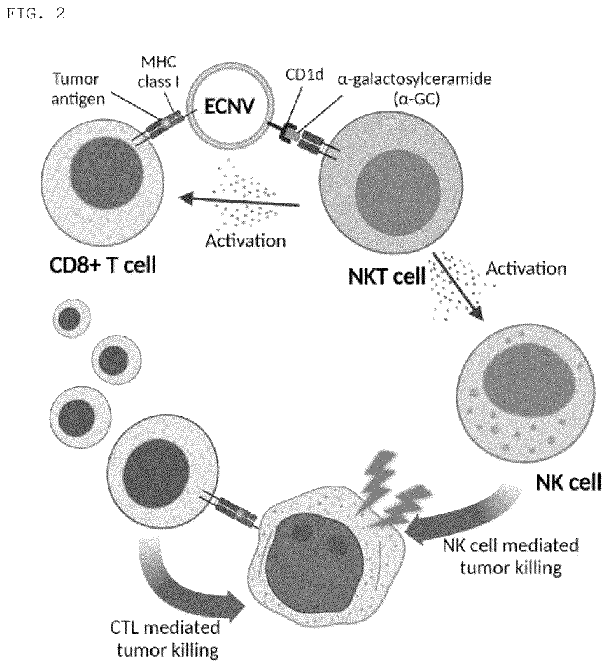

FIG. 3 illustrates experimental results of in-vitro natural killer T cell activation of a vaccine in Example 1;

FIG. 4 illustrates experimental results of in-vitro antigen-specific T cell activation of the vaccine in Example 1;

FIG. 5 illustrates experimental results of in-vivo natural killer T cell activation of the vaccine in Example 1;

FIGS. 6 and 7 illustrate experimental results of in-vivo anticancer effects of the vaccine in Example 1 using mice;

FIG. 8 illustrates results confirming the production and features of C1498-derived ECNV-αGC in Example 2;

FIG. 9 illustrates experimental results of in-vitro activation of natural killer T cell and antigen-specific T cell by an AML vaccine in Example 2;

FIG. 10 illustrates in-vivo cancer treatment and prevention effects of the AML vaccine in Example 2;

FIG. 11 illustrates results of experimental identification of anticancer efficacy-mediated core immune cells by the AML vaccine in Example 2;

FIG. 12 illustrates experimental results of identification of immunological mechanism of the AML vaccine in Example 2;

FIG. 13 illustrates experimental results of assessment of synergistic efficacy of the AML vaccine in Example 2 and chemotherapy; and

FIG. 14 illustrates preliminary experimental results of applicability of the AML vaccine in Example 2 to a human body.

### DETAILED DESCRIPTION OF THE INVENTION

The present invention provides a natural killer T cell ligand-loaded extracellular nanovesicle (NKT Ligand-ECNV) derived from cancer cells.

In addition, the present invention provides an autologous anticancer vaccine including the NKT Ligand-ECNV against blood cancer.

Further, the present invention provides an autologous anticancer vaccine against blood cancer, which enables T cell activation and includes cancer cell-derived extracellular nanovesicles (ECNV), wherein the natural killer T cell ligand (NKT Ligand) as an adjuvant is bound to the surface thereof, whereby it may recognize a cancer antigen specific to a blood cancer patient thus to have cancer cell-specific anticancer function.

Furthermore, the present invention provides a method for treatment of hematologic malignancies, which includes administering NKT Ligand-ECNV to an individual.

The natural killer T cell ligand-loaded extracellular nanovesicle (NKT Ligand-ECNV) derived from cancer cells may be produced by a process including: (i) incubating cancer cells in a medium including NKT Ligand added thereto in order to load NKT Ligand onto the cancer cells; (ii) nano-converting the NKT Ligand-loaded cancer cells by a cell extrusion apparatus; and (iii) isolating the NKT Ligand-ECNV.

In the step (i), the cancer cells may be blood cancer cells. The cancer cells may include, for example, acute myeloid leukemia cells, acute lymphoblastic leukemia cells, chronic myeloid leukemia cells, chronic lymphoblastic leukemia cells, multiple myeloma cells or T-cell lymphoma cells.

The cancer cells may be cancer cells derived from an individual who receives administration of an anticancer vaccine, or cancer cells derived from other individuals. The autologous cancer cell may be a cancer cell derived from an individual suffering from any one disease selected from the group consisting of: acute myeloid leukemia; acute lymphoblastic leukemia; chronic myeloid leukemia; chronic lymphoblastic leukemia; multiple myeloma; B-cell lymphoma; and T-cell lymphoma.

The appropriate number of cancer cells may be seeded in a medium. In one embodiment, the cancer cells may be seeded in 1×10⁵ to 1×10⁷ cells/ml of culture media. Seeding in the above range is suitable for preparation of the extracellular nanovesicles. Further, if it is beyond the above range, the membrane may tear or become damaged during the extrusion process.

The cancer cell may be inoculated at varying concentrations according to types of blood cancer cells.

The NKT Ligand may function as an adjuvant.

The NKT Ligand may be any one selected from the group consisting of alpha-galactosyl ceramide, alpha-glucuronosyl ceramide, phosphatidylinositol tetramannoside, isoglobo trihexosyl ceramide, ganglioside GD3, phosphatidylcholin, beta-galactosyl ceramide, lipophosphoglycan, glycoinositol phospholipid, beta-anomer galactosyl ceramide and alpha-anomer galactosyl ceramide.

An added concentration of the NKT Ligand is not limited to a specific range. In one embodiment, for the number of cancer cells/ml, the NKT Ligand may be added in 0.1 to 10 μg/ml to the culture media. If the NKT Ligand is added in an amount of less than 0.1 μg/ml, it may not be effectively loaded on the surface of the cancer cell. On the other hand, when the NKT Ligand is added in an amount of more than 10 μg/ml, the cancer cells may die due to toxicity.

The added concentration of the NKT Ligand may vary depending on types of cancer cells and types of the NKT Ligand.

The cell culture does not need to be necessarily conducted for a specific period of time as long as it is implemented for a time required for binding the NKT Ligand to the surface of the cell. In one embodiment, culturing may be performed for 12 to 120 hours.

When the cancer cell is cultured in the NKT Ligand added media, the NKT Ligand is loaded (bound) to the cancer cell surface receptor. Alpha-galactosyl ceramide (αGC) is bound to CD1d receptor of the blood cancer cell.

In the step (ii), the cell extrusion apparatus refers to an apparatus for extruding cells to give extracellular vesicles of nano-sized cells. For example, it may be an apparatus provided with a membrane having nano- or micro-sized pores.

The cancer cell before extrusion may include different types of extracellular vesicles (EVs). The extracellular vesicles are various types of particles secreted from the cell. Exosomes derived from an endosomal pathway, and microvesicles derived from a plasma membrane are included. The exosome contains diverse information such as a protein of a blast cell, DNA, etc. Further, the microvesicle is one of cell organelles generally having a size of 0.03 to 1 μm, which is naturally free from a cellular membrane of the cell and has the form of phospholipid bilayer. Further, it contains intracytoplasmic components such as mRNA, DNA, protein, etc.

In one embodiment, the membrane may have a pore size of 1 to 5 μm.

The NKT Ligand-ECNV obtained in the step (iii) is an extracellular nanovesicle of the cell conjugated with NKT Ligand. The ECNV nano-converted through the cell extrusion process in the step (ii) may be obtained through a process such as centrifugation or the like.

The NKT Ligand-ECNV may have, for example, a size of 100 to 1000 nm, 100 to 700 nm, 100 to 500 nm, 200 to 500 nm, or 300 to 400 nm.

In the present invention, the blood cancer may be any one selected from the group consisting of acute myeloid leukemia (AML), acute lymphoblastic leukemia (ALL), chronic myeloid leukemia (CML), chronic lymphoblastic leukemia (CLL), multiple myeloma (MM), B-cell lymphoma (BCL) and T-cell lymphoma (TCL).

The autologous anticancer vaccine of the present invention may be used in combination with any one selected from a chemotherapy, a target therapeutic agent and an immune checkpoint inhibitor.

Hereinafter, the present invention will be described in detail by means of examples.

### EXAMPLE

### Example 1: Anticancer Effects on Cellular Lymphoma and Multiple Myeloma

### (1) Production of E.G7 and MOPC315.BM-Derived ECNV and αGC Loading

As a T-cell lymphoma cell line, E.G7 cells in 1.0×10⁶ cells/ml were seeded in a culture medium including 1 μg/ml of alpha-galactosyl ceramide (αGC), followed by incubation for 24 hours. By washing the product twice in phosphate buffered saline (PBS), αGC not loaded in the tumor cell was removed. Then, the residue was suspended in PBS to reach 1.0×10⁷ cells/ml. This was subjected to extrusion eleven (11) times using a mini-extruder (Avanti Polar Lipids), followed by adding the equal amount of 0.2M sodium acetate at pH 5.2. After 30 minutes of incubation at room temperature, 2.6% 2M TRIS-HCl of the total volume of the solution was added. By centrifugation at 27,000G and 4° C. for 30 minutes, the resulting pellets were suspended in PBS or FBS-added RPMI 1640. The product was designated as αGC-E.G7 ECNV.

As a multiple myeloma cell line, MOPC315.BM-derived ECNV was also prepared by the same procedures as describe above. However, the incubation was conducted with a medium containing 1 μg/ml of αGC per tumor cells in 0.5×10⁶ cells/ml for 24 hours. The product was designated as αGC-MOPC315.BM ECNV.

### (2) Experiment for In-Vitro Activation of Natural Killer T Cells

In order to confirm whether αGC-E.G7 ECNV and αGC-MOPC315.BM ECNV can activate DN32. D3 cells, a hybridoma cell line of natural killer T cells, the following process was carried out. As a control, αGC non-loaded ECNV, that is, E.G7 ECNV and MOPC315.BM ECNV were used.

αGC-E.G7 ECNV and αGC-MOPC315.BM ECNV were cultured along with DN32. D3 cells for 24 hours. Thereafter, the IL-2 concentration as an activation indicator of natural killer T cell hybridoma was determined by sandwich ELISA assay in the culture solution.

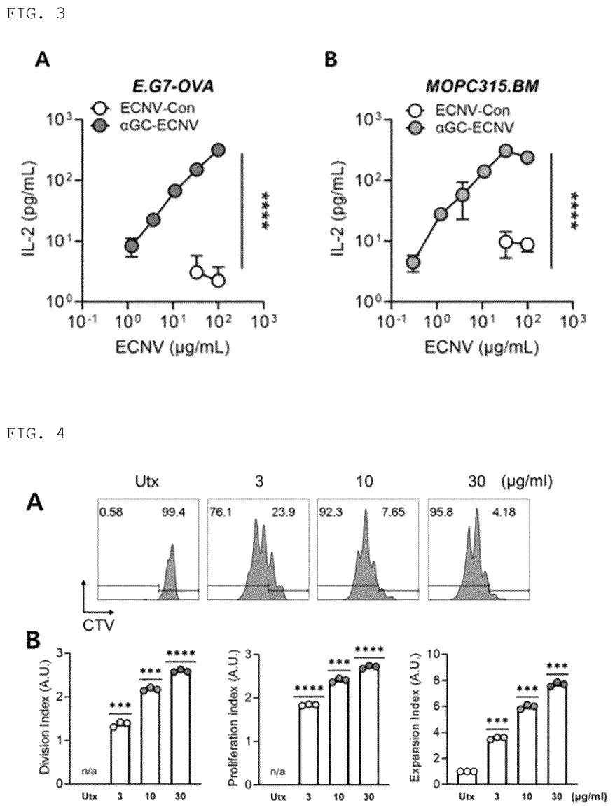

A of FIG. 3 shows experimental data of αGC-E.G7 ECNV and E.G7 ECNV, while B of FIG. 3 shows experimental data of αGC-MOPC315.BM ECNV and MOPC315.BM ECNV. Unlike ECNV-Con group with very little generation of IL-2 even at a high dose, it was confirmed that αGC-ECNV and the cultured DN32. D3 cells generate IL-2 in a concentration-dependent manner.

### (3) Experiment for In-Vitro Activation of Antigen Specific T Cells

Activation of antigen-specific CD8+ T cell by an antigen (OVA₂₅₇₋₂₆₄) inherent in αGC-E.G7 was investigated as follows.

Splenocytes of OT-I mice, which are specific to OVA₂₅₇₋₂₆₄, were obtained, and then CD8+ cells were isolated using anti-CD8 microbeads (Miltenyibiotec) after ACK lysis. Then, the isolated cells were diluted in PBS with an appropriate concentration, followed by incubation at 37 ºC for 15 minutes to label the cells with a proliferation tracking dye, Cell Trace Violet (CTV). Following this, the obtained product was washed three times with FBS 10% RPMI medium to remove CTV residue. OT-I CD8+ cells labeled with CTV were cultured along with αGC-E.G7 ECNV (αGC-ECNV) at varying concentrations for 72 hours.

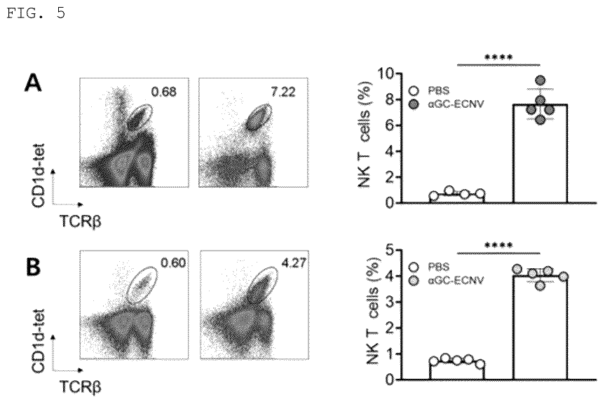

As a result of measuring the division of OT-I cell by a flow cytometer, it was observed that cell division occurred in αGC-ECNV concentration-dependent manner (A of FIG. 4). From this result, it could be confirmed using a variety of arbitrary indicators that significant cell division occurs depending on αGC-ECNV concentration (B of FIG. 4).

### (4) Experiment for In-Vivo Activation of Natural Killer T Cells

In-vivo activation of αGC-E.G7 ECNV and αGC-MOPC315.BM ECNV within natural killer T cells was investigated as follows.

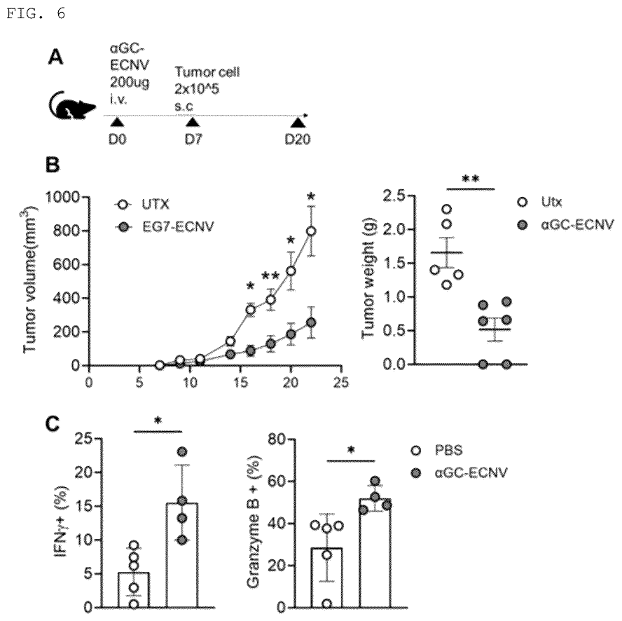

200 μg of αGC-E.G7 ECNV was suspended in 300 μL PBS and then injected into a tail vein of C57BL/6 mouse (n=5). PBS was used as a control. After 3 days, the spleen of the mouse was taken and subjected to ACK lysis, followed by flow cytometry analysis. As a result, it was confirmed that CD1d-tet+TCRb+ NKT cells are significantly increased by αGC loaded in ECNV (A of FIG. 5).

Further, 200 μg of αGC-MOPC315.BM ECNV was suspended in 300 μL PBS, and then injected into a tail vein of ALB/C mouse (n=5). PBS was used as a control. After 3 days, the spleen of the mouse was taken and subjected to ACK lysis, followed by flow cytometry analysis. As a result, it was confirmed that CD1d-tet+TCRb+ NKT cells were significantly increased by αGC loaded in ECNV (B of FIG. 5).

### (5) Experiment for In-Vivo Anticancer Effects Using Mice

200 μg of E.G7-OVA derived αGC-E.G7 ECNV was suspended in 300 μL PBS, and then injected into a tail vein of C57BL/6 mouse. At day 7 after immunization, the same cancer cell line in 2.0×10⁵ cells was suspended in 100 μL PBS, and then, subcutaneously (s.c.) injected in the left flank of the mouse. Once the tumor was visible, the size of the cancer was measured every 2 to 3 days. After 20 days, the cancer was collected, and lymphocytes in the tumor were isolated, followed by determining the generation of cytokine through a flow cytometer.

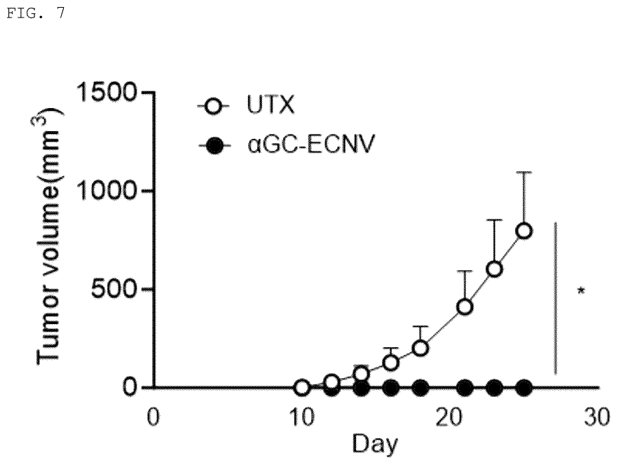

The experiment was conducted using five (5) animals in 8-week old female mice control (PBS), as well as six (6) animals in an experimental group (αGC-ECNV). As a result of the experiment, it was observed in A of FIG. 6 that the cancer was not generated in two (2) out of the six (6) animals administered with αGC-E.G7 ECNV (αGC-ECNV). From day 16 after cancer cell injection, significant cancer generation rate was observed in both of the groups. Further, as a result of extracting the cancer at day 21 and weighing the same, it was confirmed that the size of the cancer was significantly suppressed in the αGC-ECNV group (B of FIG. 6).

In CD8 T cells within the tumor of the mice immunized with αGC-ECNV, it was confirmed that the generation of cytotoxic cytokines such as granzyme B and IFNg is higher than the control mice (C of FIG. 6).

According to the same procedures as used for αGC-E.G7 ECNV, 200 μg of αGC-MOPC315.BM ECNV was suspended in 300 μL PBS, and then injected into a tail vein of C57BL/6 mouse. At day 7 after immunization, 2.0×10⁵ cells of the same cancer cell line was suspended in 100 μL PBS, and then, s.c. injected in the left flank of the mouse. Once the tumor was visible, the size of the cancer was measured every 2 to 3 days.

With regard to five (5) animals in 8-week old female mice control (PBS), as well as five (5) animals in the experimental group (αGC-ECNV), it was observed that tumor was generated in four (4) out of the five (5) animals in the control, whereas all of the five (5) animals in the experimental group did not show tumor (FIG. 7).

### Example 2: Anticancer Effects on Acute Myeloid Leukemia (AML)

### (1) Production of C1498 Derived ECNV-αGC and Confirmation of Features

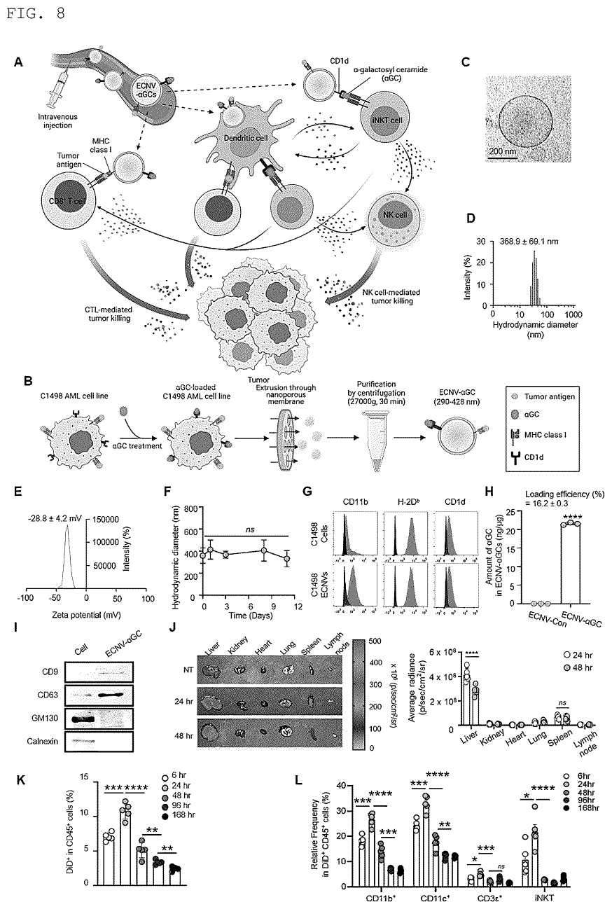

A of FIG. 8 is a schematic view illustrating the mechanism of action of the vaccine in Example 2. When the vaccine enters the body through intravenous (i.v.) injection, different types of immune cells such as CD8 T cells, dendritic cells, natural killer T cells, and the like are activated.

As shown in B of FIG. 8, for a mouse AML cell line, that is, C1498 cells in 1.0×10⁶ cells/ml, the cells were cultured for 24 hours using a medium containing 1 μg/ml of alpha-galactosyl ceramide (αGC). The cultured product was washed twice with phosphate buffered saline (PBS) to remove αGC that has not been loaded to the tumor cells. Then, the product was suspended in PBS to reach 1.0×10⁷ cells/ml. After mounting a 1 μm sized filter on a mini-extruder (Avanti Polar Lipids), the above solution passes through the filter 11 times to proceed extrusion, followed by adding pH 5.2 0.2M sodium acetate in equal amount. After leaving the mixture at room temperature for 30 minutes, 2.6% of the total volume of 2M TRIS-HCl was added. By centrifugation at 27,000G and 4° C. for 30 minutes, the resulting pellets were suspended in PBS or FBS-added RPMI 1640 thus to produce AML vaccine. This was designated as αGC-C1498 ECNV.

Cryo-EM image of αGC-C1498 ECNV was monitored through an electron microscope (C of FIG. 8), while measuring a size thereof through DLS assay (D of FIG. 8). The size of αGC-C1498 ECNV was 358±69.1 nm. Further, zeta potential of αGC-C1498 ECNV was measured (E of FIG. 8) and zeta potential stability was confirmed (F of FIG. 8). αGC-C1498 ECNV was stably present in the form of nanoparticles for 8 days without aggregation in the serum.

As a result of flow cytometry analysis for surface molecules of the vaccine as well as cancer cell line, like C1498 cells, αGC-C1498 ECNV has maintained MHC class I (H2-db) and CD1d without any change (G of FIG. 8). MHC Class I is a receptor to which the antigen can be bound, and CD8 T cell may recognize such MHC-I:antigen complex to be activated. CD1d may also become a complex along with αGC, thereby activating natural killer T cells. Accordingly, to preserve the surface receptors is important for anticancer effects of αGC-C1498 ECNV.

An amount of αGC loaded in αGC-C1498 ECNV was determined (H of FIG. 8).

The purity of αGC-C1498 ECNV was evaluated by Western blot. As shown in I of FIG. 8, αGC-C1498 ECNV expressed exosomal markers such as CD9, CD63, etc., while not containing cell organells debris such as GM130, Calnexin, etc.

By administering αGC-C1498 ECNV, its distribution in the entire body and major organs of the mouse was investigated. More particularly, after applying a fluorescent material DiR to αGC-C1498 ECNV, the ECNV was i.v. injected into the mouse. After several hours, some organs (heart, lung, marrowbone, liver, kidney and spleen) were excised and subjected to determination of fluorescence intensity. αGC-C1498 ECNV existed in the liver, an organ where NKT cells are most abundant, and the spleen as a secondary lymphoid organ 48 hours post administration (J of FIG. 8). αGC-C1498 ECNV interacted with and/or were taken up by CD45+ immune cells by 24 hours after administration (K of FIG. 8), and further interacted with major immune cell subsets including antigen presenting cells expressing CD11b or CD11c (L of FIG. 8).

### (2) Confirmation for In-Vitro Activation of Natural Killer T Cells and Antigen-Specific T Cells

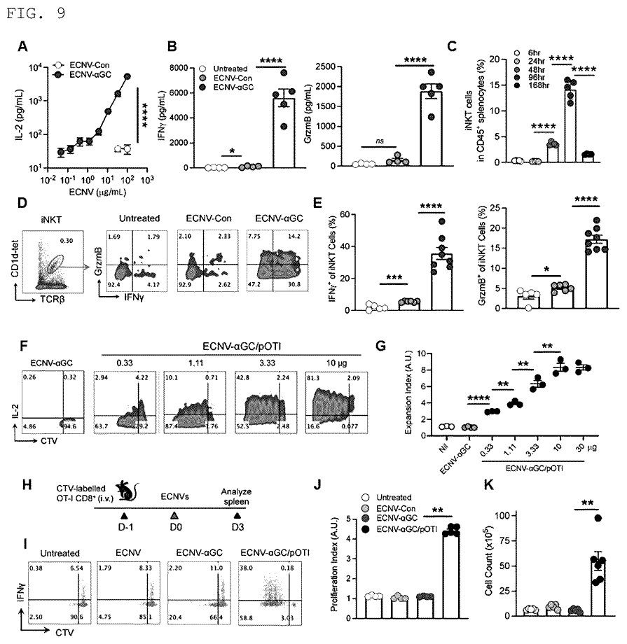

αGC-C1498 ECNV at a predetermined concentration was co-cultured with mouse NKT hybridoma cell line, DN32. DN for 24 hours. Further, as a control, C1498-derived ECNV without loading of αGC (“αGC non-loaded”) was used. As a result, in the control, IL-2 was not secreted even at a high concentration. On the other hand, it was confirmed that the experimental group (αGC-C1498 ECNV) shows concentration-dependent secretion of IL-2 cytokine (A of FIG. 9).

Further, 6 hours after i.v. administration of 200 μg of αGC-C1498 ECNV to the mouse, the serum was separated and subjected to ELISA experiment. From the experiment, it was confirmed that cytolytic molecules such as IFNγ and Granzyme B, etc. were significantly secreted (B of FIG. 9). Further, several hours after i.v. injection of αGC-C1498 ECNV into the mouse, the spleen was excised and subjected to flow cytometry analysis. As a result of the analysis, it was confirmed that NKT cells were vigorously proliferated (C of FIG. 9).

As a result of flow cytometry analysis for NKT cells of the mouse administered with αGC-C1498 ECNV, it was confirmed that secretion of cytolytic molecules such as IFNγ and Granzyme B, etc., was significantly increased compared to the control (D and E of FIG. 9).

In order to investigate whether αGC-C1498 ECNV activates antigen-specific CD8 T cells, OVA₂₅₇₋₂₆₄ peptide was combined during production of the vaccine. Then, CTV dye (FACS dye capable of determining cell division) was applied to OT-I CD8 T cells specifically responding to the above peptide/antigen, followed by co-culturing with the vaccine at varying concentrations. As a result of flow cytometry analysis, it was confirmed that OT-I CD8 T cell division and IL-2 secretion are controlled in concentration-dependent manner (F and G of FIG. 9).

24 hours after i.v. administration of 2×10⁶ CTV-labeled naive OT-I CD8⁺ T cells to WT mouse, peptide (OT-I)-loaded αGC-C1498 ECNV was i.v. injected into the mouse, the spleen was excised after 3 days. As a result of flow cytometry analysis, cell division was observed only in the mice administered with αGC-C1498 ECNV (I, J and K of FIG. 9).

### (3) Determination of In-Vivo AML Treatment and Preventive Effects

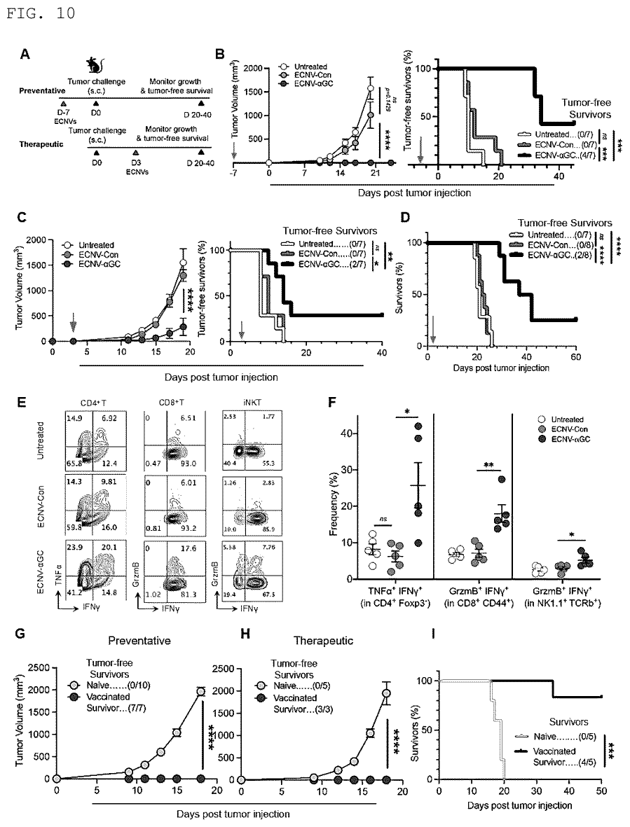

In order to investigate the anti-leukemia efficacy of αGC-C1498 ECNV, AML preventive model and treatment model were constructed (A of FIG. 10). Mice of the above two models were subjected to subcutaneous administration of AML cell line C1498 cells.

With regard to the preventive model, there were no surviving mouse in a group with no administration (Untreated) and another group with administration of αGC non-loaded ECNV (ECNV-Con). On the other hand, the αGC-C1498 ECNV administered group (ECNV-αGC) showed more than half of the animals surviving 40 days or more (B of FIG. 10).

With regard to the treatment model, like the preventive model, there were no surviving mouse in a group with no administration (Untreated) and another group with administration of αGC non-loaded ECNV (ECNV-Con). On the other hand, a group administered with αGC-C1498 ECNV (ECNV-αGC) showed that about 28% of the animals survived for 18 days or more (FIG. 10). Further, in a cancer treatment model wherein C1498 cells were i.v. administered and, after 2 days, αGC-C1498 ECNV was administered, a group administered with AML vaccine also showed significantly improved survival rate (D of FIG. 10).

With regard to the treatment model, cancer was introduced and, after 14 days, the cancer was excised and TIL was isolated therefrom. As a result of flow cytometry analysis, it was confirmed that the generation of cytolytic molecules such as TNFa, Gzmb, IFNγ, etc. was significantly higher in CD4, CD8, NK cells of αGC-C1498 ECNV administered mice (E of FIG. 10). The surviving mice in B and C of FIG. 10 were gathered and then subjected to a rechallenge experiment in which the same cancer was inoculated again. As a result of the experiment, it was confirmed that the cancer was not generated in the αGC-C1498 ECNV-administered mice and exhibited a significant improvement in survival rate (G, H and I of FIG. 10). These results collectively demonstrates that immune memory was established in the αGC-C1498 ECNV-administered mice.

### (4) Identification of Anticancer Efficacy-Mediated Core Immune Cells by αGC-C1498 ECNV

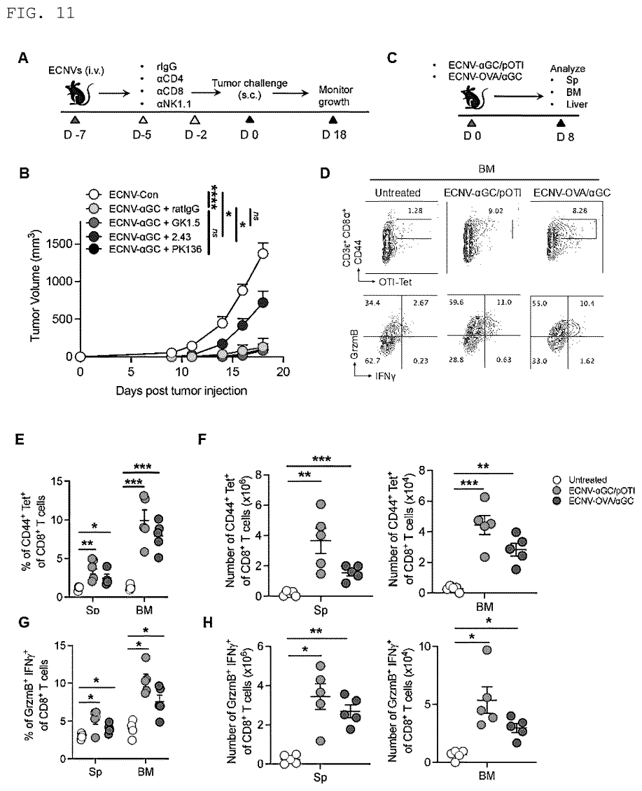

In order to identify the mechanism of cancer-specific T cell generation by αGC-C1498 ECNV, the preventive model used previously was used again. Particularly, after AML vaccination, antibodies for depletion of CD4+ T cell (GK1.5), CD8+ T cell (2.43) and NK cell (PK136) were administered, followed by observing tumor growth (FIG. A of FIG. 11).

In the mice depleted of CD4 T cell and NK cell, anticancer efficacy of αGC-C1498 ECNV was still observed. On the other hand, the mice depleted of CD8 T cell has considerably reduced anticancer efficacy and showed a cancer growth pattern similar to the mice administered with the control vaccine (B of FIG. 11). It could be understood that the anticancer efficacy of αGC-C1498 ECNV is dependent of CD8 T cells.

AML vaccine combined with peptide or produced using C1498 cell line that expresses OVA was administered. Thereafter, antigen-specific CD8 T cell by the vaccine was analyzed through H-2K^{b}OVA₂₅₇₋₂₆₄ tetramer staining in different organs (C of FIG. 11). As a result, it was confirmed that both of the vaccine combined with peptide and the vaccine produced using OVA-expressing C1498 cell line showed significant increase in antigen-specific effector CD8 T cell, Gzmb and IFNγ secretion, as compared to Untreated mice (D and H of FIG. 11).

### (5) Experiment for Identification of Immunological Mechanism of AML Vaccine

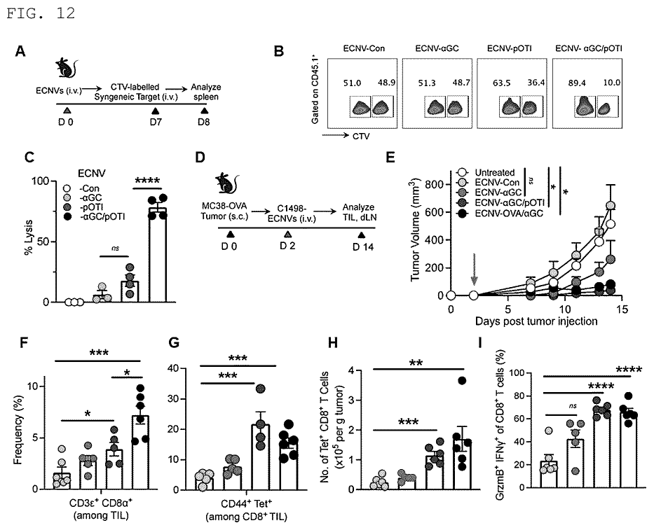

To study the features and mechanism of cancer antigen-specific CD8 cell activation by αGC-C1498 ECNV, a control vaccine (ECNV-Con), αGC-only loaded AML vaccine (ECNV-αGC), POT-I-only loaded AML vaccine (ECNV-pOTI), and αGC and pOTI-loaded AML vaccine (ECNV-αGC/pOTI) were each i.v. administered to a mouse. After 7 days, CTV-labeled spleen cells of CD45.1 mouse, which can be distinguished by a congenic marker, were also i.v. injected. After 18 to 21 hours, the spleen was excised, followed by flow cytometry analysis (A of FIG. 12). As a result, the most efficient antigen-specific target cell lysis was confirmed in the case of ECNV-αGC/pOTI. Further, ECNV-POTI also demonstrated in vivo CTL response (B and C of FIG. 12). Accordingly, it could be understood that ECNV having αGC loaded on the surface may efficiently accelerate proliferation of antigen-specific CD8 T cells and differentiation thereof into cytotoxic effector.

After subcutaneous injection of a different type of cancer cells (MC38-OVA) expressing the cognate antigen OVA, a variety of C1498-derived vaccines was i.v. injected. After 14 days, cancer was excised, followed by flow cytometry analysis (D of FIG. 12). As compared to ECNV-Con, administration of αGC-only loaded AML vaccine (ECNV-αGC) and pOT-I-only loaded AML vaccine (ECNV-POTI) slightly inhibits MC38-OVA tumor growth. On the other hand, AML vaccine loaded with both of αGC and pOTI (ECNV-αGC/pOTI), as well as AML cell-derived vaccine including transfection of αGC and OVA (ECNV-OVA/αGC), almost completely inhibited the same tumor growth (E of FIG. 12).

Further, when cancer-infiltrated cells were subjected to flow cytometry analysis, it was confirmed that the CD8 cell rate in mice administered with ECNV-αGC/POTI and ECNV-OVA/αGC, and the antigen-specific effector cell (CD44+Tet+) frequencies and absolute number are significantly high (F, G, H and I of FIG. 12).

### (6) Experiment for Assessment of Synergistic Efficacy of AML Vaccine and Chemotherapy

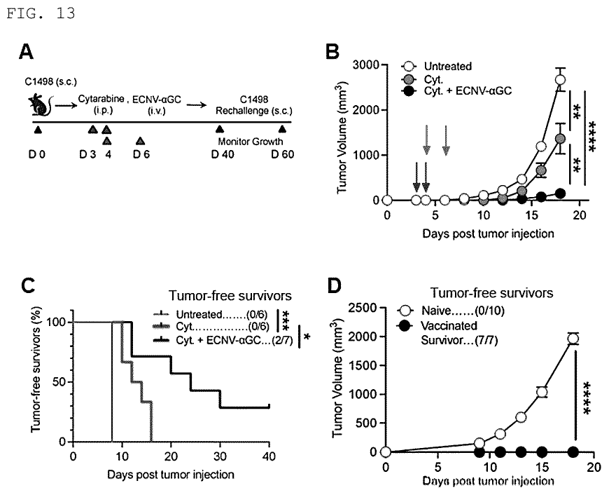

In order to assess synergistic efficacy with cytarabine, which is used as an AML therapeutic agent in clinical phase, C1498 cell line was subcutaneously injected and, after 3 days and 4 days, followed by intraperitoneal (i.p.) injection of cytarabine. Then, after 4 days and 6 days, αGC-C1498 ECNV was additionally administered. Whether recurrence of C1498 can be delayed with αGC-C1498 ECNV, was monitored in terms of tumor growth and survival (A of FIG. 13).

As a result, when using cytarabine in combination with αGC-C1498 ECNV rather than treatment using cytarabine alone, it was confirmed that cancer generation is remarkably suppressed (B of FIG. 13). Particularly, there was no surviving mouse in the group administered with cytarabine alone, whereas the group administered with cytarabine in combination with αGC-C1498 ECNV showed that 2 out of 7 animals survived for 40 days or more without the generation of cancer (C of FIG. 13). When the same cancer was transplanted to the surviving mouse, it was confirmed that the rechallenged mice did not have cancer generation and exhibited significant survival rate (D of FIG. 13).

### (7) Pre-Experiment for Applicability to Human Body of AML Vaccine

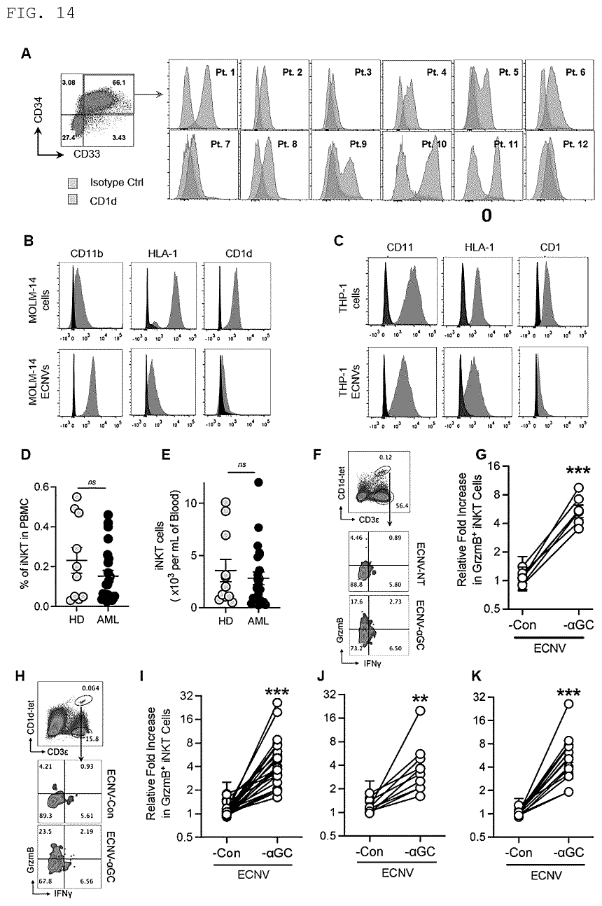

In order to investigate whether AML vaccine exhibits medical effects in humans, human AML cell lines, MOLM-14 and THP-1 were loaded with αGC, followed by the production of AML vaccine according to the method described above (αGC-MOLM-14 ECNV and αGC-THP-1 ECNV). Like each cell, αGC-MOLM-14 ECNV and αGC-THP-1 ECNV were also determined to preserve CD1d and MHC-Class I (B and C of FIG. 14).

As a result of flow cytometry analysis for NKT cells in the blood of 12 patients with acute myeloid leukemia, it was confirmed that all of the cells express CD1d (A of FIG. 14).

It was confirmed that NKT % and absolute number in both of AML patients and healthy persons are not significantly different from each other (D and E of FIG. 14). αGC-MOLM-14 ECNV was co-cultured with the blood of a healthy person, followed by flow cytometry analysis. Within NKT cells, it was confirmed that PBMC cultured with αGC-loaded vaccine significantly and largely expressed Gzmb and IFNγ, compared to PBMC cultured with non-loaded vaccine (F and G of FIG. 14).

αGC-THP-1 ECNV was co-cultured with the blood of AML patient, followed by flow cytometry analysis. Within NKT cells, it was confirmed that PBMC cultured with αGC-loaded vaccine significantly and largely expressed Gzmb and IFNγ, compared to PBMC cultured with non-loaded vaccine (FIGS. 14H and 14I). Like the mice, a human AML vaccine can efficiently activate NKT cells, which suggests the possibility of expressing anticancer activity in humans.

What is claimed is:

1. A method for treatment of hematologic malignancies (“blood cancer”), comprising administering cancer cell-derived extracellular nanovesicle (ECNV) to an individual, wherein natural killer T cell ligand is bound to a surface of the ECNV.

2. The method according to claim 1, wherein the natural killer T cell ligand is any one selected from the group consisting of alpha-galactosyl ceramide, alpha-glucuronosyl ceramide, phosphatidylinositol tetramannoside, isoglobo trihexosyl ceramide, ganglioside GD3, phosphatidylcholin, beta-galactosyl ceramide, lipophosphoglycan, glycoinositol phospholipid, beta-anomer galactosyl ceramide and alpha-anomer galactosyl ceramide.

3. The method according to claim 1, wherein the cancer cell is derived from a patient suffering from any one disease selected from the group consisting of acute myeloid leukemia (AML), acute lymphoblastic leukemia (ALL), chronic myeloid leukemia (CML), chronic lymphoblastic leukemia (CLL), multiple myeloma (MM), B-cell lymphoma (BCL) and T-cell lymphoma (TCL).

4. The method according to claim 1, wherein the blood cancer is any one selected from the group consisting of acute myeloid leukemia (AML), acute lymphoblastic leukemia (ALL), chronic myeloid leukemia (CML), chronic lymphoblastic leukemia (CLL), multiple myeloma (MM), B-cell lymphoma (BCL) and T-cell lymphoma (TCL).

5. The method according to claim 1, wherein the extracellular nanovesicle includes a cancer antigen.

6. The method according to claim 1, wherein the natural killer T cell ligand is bound to CD1d receptor on the surface.

7. The method according to claim 1, wherein the cancer cell is an autologous cancer cell derived from the individual.

8. The method according to claim 1, wherein the extracellular nanovesicle is an exosome or micro-vesicle having a size of 100 to 1,000 nm.

9. The method according to claim 1, further comprising administering any one selected from the group consisting of a chemotherapy, a target therapeutic agent and an immune checkpoint inhibitor in combination with the ECNV.
